(19) United States
(12) Patent Application Publication
(10) Pub. No.: US 2007/0042091 A1
(43) Pub. Date: Feb. 22, 2007

(54) Apparatus and Method for Determining the Amount of Time Remaining to Cook Food to a Desired Temperature

(75) Inventors: Richard Rund, Great Falls, VA (US); Pierre Charlety, Deng Chau, Hong Kong (CN); Ma Huai Wang, Zhaoqing City, Guangdong Province (CN); Quan Zhi Yong, RongCheng Town, HeNan Province (CN)

Correspondence Address: CAHN & SAMUELS LLP, 2000 P STREET NW, SUITE 200, WASHINGTON, DC, 20036, US

(73) Assignee: F.O.B. INSTRUMENTS, LTD., Great Falls, VA (US)

(21) Appl. No.: 11/428,260

(22) Filed: Jun. 30, 2006

Related U.S. Application Data
(60) Provisional application No. 60/780,830, filed on Mar. 10, 2006.
(60) Provisional application No. 60/709,446, filed on Aug. 19, 2005.

(51) Int. Cl. G01N 33/02

(52) U.S. Cl. 426/231

(57) ABSTRACT

A method and an apparatus capable of determining the amount of time remaining before a food being cooked reaches a desired temperature.

This application claims the benefit of U.S. provisional application Serial No. 60/709,446, filed Aug. 19, 2005, and claims the benefit of U.S. provisional application Ser. No. 60/780,830, filed Mar. 10, 2006, which are incorporated herein by reference.

### FIELD OF THE INVENTION

This invention relates to the calculation of the time remaining before a desired temperature is reached for a food being cooked.

### BACKGROUND OF THE INVENTION

Most food safety recommendations are for cooking foods to particular temperatures based on the food itself. This requires that the user periodically check the temperature of the food being cooked to see if the desired temperature has been reached, and depending upon the recommendation being used begin a timer for a period of time while keeping the food at the particular temperature for that time period.

Most recipes on the other hand are for cooking a food for a particular time period in an environment of a certain temperature on the theory that the food being cooked will reach the needed temperature shortly before or at the expiration of the time period. That is not a problem unless the oven or other cooking device does not provide the correct cooking temperature or the user does not provide sufficient heat to the food, for example, during stove top cooking or grilling.

A need exists for assisting the cook in providing a better indication of when a food item will reach the desired temperature while removing the guess work inherent in most recipes and not easily predicted under the food safety recommendations.

### SUMMARY OF THE INVENTION

This invention provides a method and an apparatus for determining the amount of time remaining for an item being cooked to reach a desired temperature.

At least one exemplary embodiment includes a system including a probe having a temperature sensing component; a display coupled to the thermometer circuitry; a thermometer circuitry in communication with the temperature sensing component, the thermometer circuitry having means for receiving a desired temperature, means for calculating current temperature, means for determining time remaining to cook based upon temperature rate change, and means for controlling the display to show time remaining to cook; and an interface coupled to the thermometer circuitry.

At least one exemplary embodiment includes a method for determining the time until a food reaches a desired temperature, the method including: receiving the desired temperature, determining a current temperature for the food, measuring a change in temperature as a function of time, using the temperature change to predict the time until the food reaches the desired temperature, starting a timer from the predicted time, and repeating the determining, measuring, and using steps at least one time.

At least one exemplary embodiment includes a method for determining the time until a food reaches a desired temperature, the method including: receiving the desired temperature, determining a current temperature for the food, measuring a change in temperature as a function of time, using the temperature change to predict the time until the food reaches the desired temperature, and starting a timer from the predicted time.

Given the following enabling description of the drawings, the apparatus should become evident to a person of ordinary skill in the art.

### BRIEF DESCRIPTION OF THE DRAWINGS

The aspects of the present invention will become more readily apparent by describing in detail illustrative, non-limiting embodiments thereof with reference to the accompanying drawings.

FIG. 1 illustrates an exemplary method according to the invention.

FIG. 2 illustrates an exemplary method according to the invention.

FIG. 3 illustrates an exemplary method according to the invention.

FIG. 4 illustrates an exemplary method according to the invention.

FIG. 5 depicts an exemplary display for use in an exemplary embodiment according to the invention.

FIG. 6 illustrates an exemplary display for use in an exemplary embodiment according to the invention.

FIG. 7 depicts an exemplary display for use in an exemplary embodiment according to the invention.

FIG. 8 illustrates a block diagram of an exemplary embodiment according to the invention.

### DETAILED DESCRIPTION OF THE DRAWINGS

The invention includes a method for determining the amount of time remaining before a food being cooked reaches a desired temperature. The invention also includes an apparatus for performing the method and providing that information to a user in at least one exemplary embodiment.

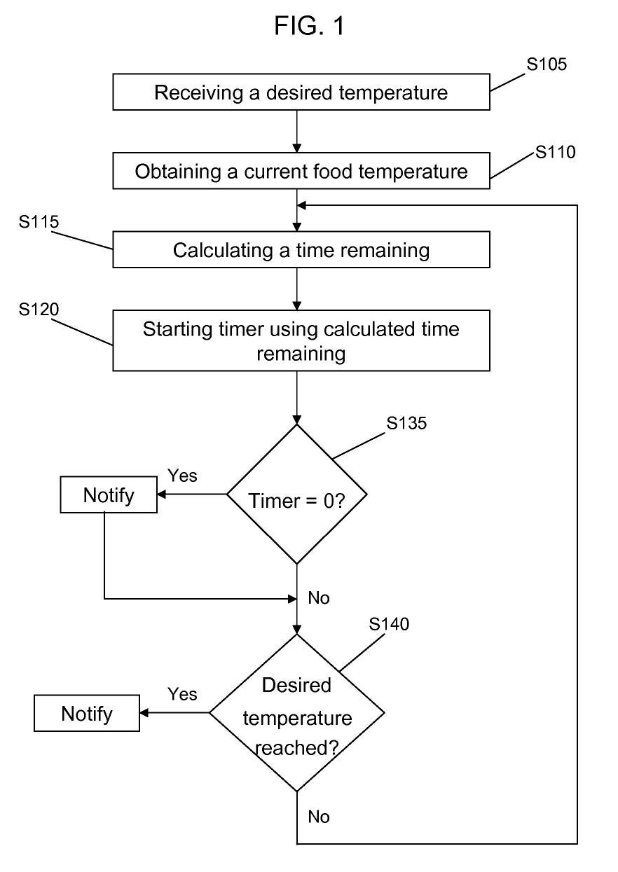

An exemplary method for determining the amount of time remaining before a desired temperature is reached for the food is illustrated in FIG. 1. The illustrated method begins with receiving a desired temperature for the food, S105, and determining (or obtaining) the current temperature of the food, S110. Steps S105 and S110 may be performed in any order or substantially concurrently with each other.

In an exemplary embodiment where it is desired to not display the temperature below a certain temperature, the display may provide a general representation of the temperature. For example, if the temperature is below 75° F., then the display shows COOL or some other indicator. Another example is when the food has a temperature between 75° F. and 100° F., the display shows WARM or some other indicator. A variety of other words may be displayed to indicate the temperature, including the temperature itself. The particular temperature ranges may be adjusted for a particular implementation.

In step S115, calculating the time it takes the food item to go from, for example, 90° F. to 100° F.; however, other temperature ranges may be used such as starting temperature to 90° F., 100° F., etc. or over the first 50°, 100°, 150°, or 20°, which could be delayed for a predetermined time or temperature. The calculated time is used to predict how long it will take the food to reach the desired temperature, S115. An exemplary formula is
t_{R}=(T_{D}−T₂)/(T₂−T₁)*(t₂−t₁)
An alternative formula is
t_{R}=(T_{D-}T₁)/(T₂−T₁)*(t₂−t₁)+t₁−t₂
In the equation, t_{R} equals the cooking time to reach the desired temperature and this is the time that is used to provide the new starting point for the timer on the display, S120. T_{D} represents the desired temperature, T₁ equals the temperature at the start of the sampling, and T₂ equals the temperature at the end of the sampling with t₁ and t₂ equaling the respective times of the temperature sampling.

Examples of the time remaining calculation includes when the starting temperature is 100° F., the end sampling temperature is 120° F. and it took 20 seconds to reach 120° F. In that example, the temperature rise is at 1° F. per second. If the desired temperature is 220° F., then the remaining time is 100 seconds. The timer will display a representation of 100 seconds, for example, “01:40” or “00:01:40” and begin the timer function. Another example is assume the next 20 second sampling period reaches an end sampling temperature of 160° F., which results in a 2° F. per second temperature rise. This temperature rise results in a remaining time of 30 seconds, and the readout for the display will be changed to 30 seconds and the timer started from 30 seconds.

When the timer reaches zero, an alarm sounds or other notification occurs to let the cook know that the food is predicted as being cooked, S135. This timer alarm/notification is superseded when the temperature reaches the desired temperature for the food, notifying the cook that the food is cooked, for example, by displaying “DONE” on the display, S140. The display may flash the exemplary word notification or display the text without flashing. Other exemplary notifications include sounding an audio alarm, flashing a visual cue such as a light, and any other type of timer notifications.

The timer displays the remaining time to reach the desired temperature. The method encompasses displaying the time as hours:minutes:seconds, minutes:seconds, and switching between the two displays at a predetermined time junction and/or allowing the cook to switch between the display options.

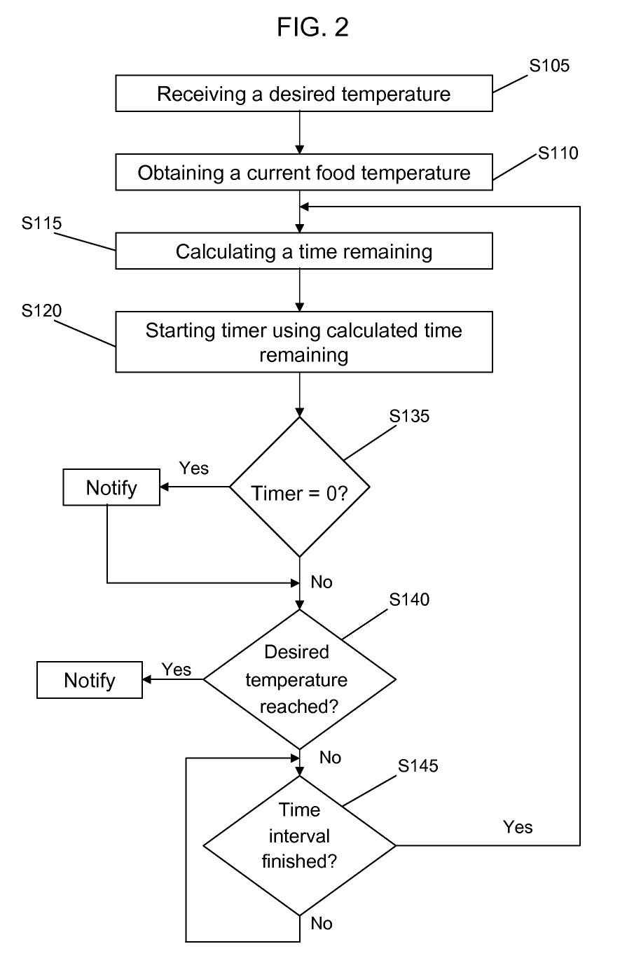

In the exemplary method illustrated in FIG. 1, steps S115 through S140 are repeated for each predetermined sampling period. T₁ equals the prior T₂ and the time differential would be the sampling period. Exemplary sampling periods are 5 seconds, 10 seconds, or 20 seconds. The sampling periods in at least one exemplary embodiment are selected from a range of 0 to 300 seconds; however, the sampling period may be any length of time. As illustrated in FIG. 2, other exemplary embodiments instead of having a continual sampling of the temperature rise perform the sampling at predetermined time intervals, S145, for example, every 5 or 10 minutes. An exemplary way to accomplish time intervals is by using counters or similar mechanisms.

Another exemplary embodiment uses a sampling period that is determined by taking a predetermined percentage of the remaining time. This calculation of sampling time based on percentage continues until the remaining time has reached a predetermined threshold such as 20 seconds or 1 minute.

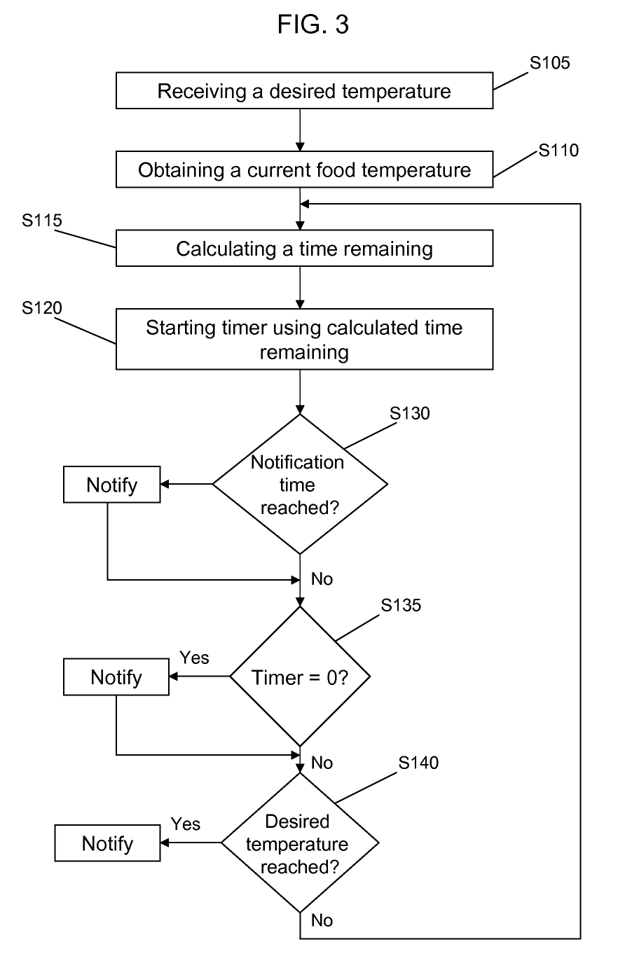

The exemplary method illustrated in FIG. 3 adds a notification to the cook at a predetermined amount of time remaining for the food to reach the desired temperature, S130. The notification typically is an audio alarm, but may be a visual cue such as a flashing light or notification on the display. An exemplary time to notify the cook is in a range of 1 to 20 minutes. In at least one exemplary embodiment the notifications occur at different times and are of different character to allow the cook to have a countdown other than the timer to the food being done, which will assist in cooking other foods and have them done at approximately the same time.

In at least one exemplary embodiment, there is a notification when the food reaches the temperature above a predetermined threshold for displaying the current temperature, when a sampling period finishes, and/or the first sampling period finishes.

In other exemplary embodiments, the sampling period is based on a predetermined change in temperature (instead of time as illustrated in FIG. 1), for example, 50°, 100°, or 20°.

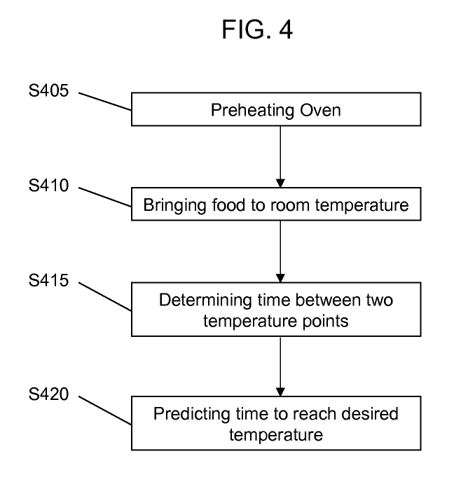

FIG. 4 illustrates an exemplary method for determining the time left begins with preheating the oven (or other enclosed cooking environment), S405. The item to be cooked is allowed to reach room temperature plus or minus a few degrees, S410. Steps S405 and S410 may be performed concurrently or reverse order. The thermometer tracks the time (starting with activation of a button to coincide with insertion of the probe into the food) it takes to go from room temperature, which it knows upon insertion of the probe into the item, to for example 100° F (or some other predetermined temperature), S415. Based on this time period, predicting the time to reach the desired temperature taking into account an accelerated pace for temperature increase based on known cooking information for the food being cooked, S420. In at least one exemplary embodiment, after the initial determination of the prediction time, sampling the temperature rise as discussed in connection with FIGs. 1-3 above.

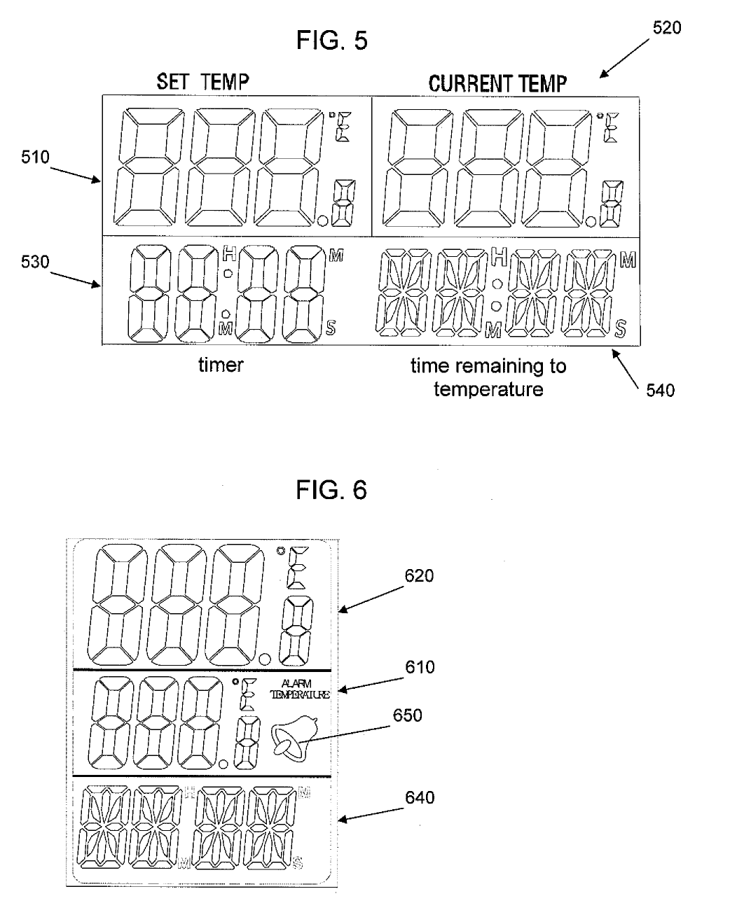

FIGS. 5-7 illustrate different exemplary display interfaces. FIG. 5 illustrates an exemplary display arrangement for displaying the desired temperature 510, the current temperature 520, a general purpose timer 530, and the time remaining until the desired temperature is reached 540. The general purpose timer, for example, could utilize the Double:Time technology offered by F.O.B. Instruments that allows the user to switch between hours:minutes to minutes:seconds. An exemplary interface includes a pair of buttons (or touch screen areas) for increasing and decreasing the value of the desired temperature setting and the timer value. In at least one exemplary embodiment, the depression of both buttons will toggle the temperature unit between Celsius and Fahrenheit; however, there could be a specific toggle button to perform this function. The exemplary interface also includes a start/stop button to deactivate any alarm/notification and operate the timer. The exemplary interface if the Double:Time technology is being used includes a button, a touch screen area, or other activation mechanism to toggle between the settings. The exemplary interfaces include an indication as to whether the temperatures are being displayed as Celsius or Fahrenheit.

FIG. 6 illustrates an exemplary display arrangement that includes the desired temperature 610, the current temperature 620, and the time remaining to cook 640. The illustrated display includes a bell image 650 that is lit as part of notification(s). The interface would be similar to that described for the display illustrated in FIG. 5 with one difference being the Double:Time technology if present would be used to switch the display mode for the time remaining to cook.

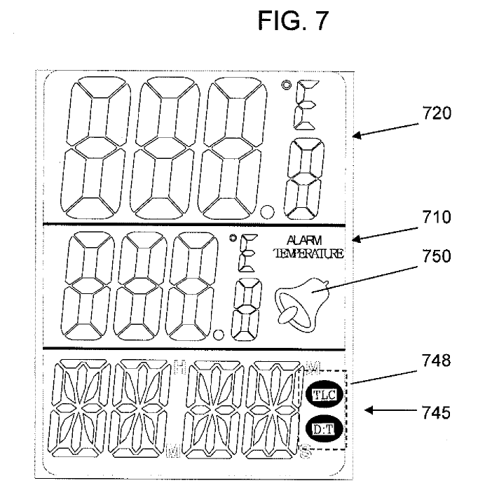

FIG. 7 illustrates an exemplary display arrangement that includes the desired temperature 710, the current temperature 720, and a timer area 745. The timer area 745 includes an indicator 748 as to whether the timer 745 is for the time remaining to reach a temperature is displayed or a general timer is displayed, which in this example the general timer is using the Double:Time technology although a generic timer could be used instead.

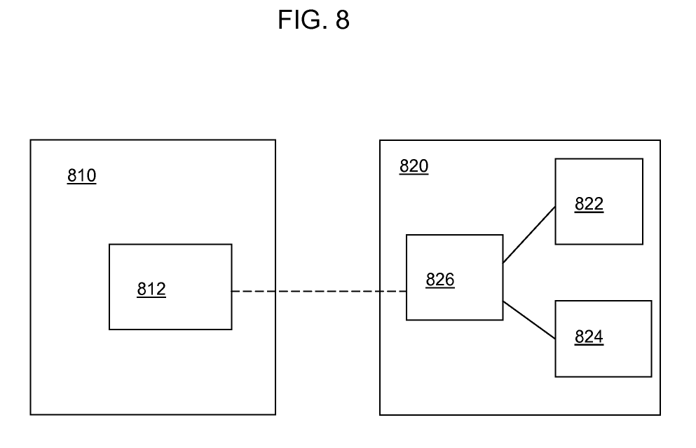

An exemplary apparatus for performing the different exemplary methods is illustrated in FIG. 8. The illustrated apparatus includes a probe 810 and a housing 820. The probe 810 and the housing 820 can be connected directly together, connected via a wire, or in wireless communication. If the probe 810 and the housing 820 are connected via a wire then it preferably is flat to lessen the impact on the seal of the oven for embodiments intended for oven use or other sealed environments. The probe 810 includes a temperature sensing component 812. The housing 820 as illustrated includes a display 822, an interface 824, and thermometer circuitry 826 in communication with the temperature sensing component 812. The interface 824 receives user input for the thermometer circuitry 826 and in some exemplary arrangements the information shown on the display 822. The thermometer circuitry 826 includes means for performing the functional steps described above in connection with any one of the different exemplary methods such that it is capable of performing the described method. In an exemplary embodiment, the working voltage for an integrated circuit that is a part of the thermometer circuitry 826 is 3 V.

An exemplary range for operation of the apparatus is in a temperature range between −40° C. to 300° C. or −40° F. to 572° F. Exemplary accuracy levels for measuring temperature are shown below:

| Temperature | Accuracy | Temperature | Accuracy |
|---|---|---|---|
| −40° C. to 0° C. | +/−2° C. | −40° F. to −32° F. | +/−4° F. |
| 0° C. to 150° C. | +/−1° C. | 32° F. to 302° F. | +/−2° F. |
| 150° C. to 177° C. | +/−2° C. | 302° F. to 350° F. | +/−4° F. |
| other ranges | +/−4° C. | other ranges | +/−8° F. |

Those having ordinary skill in the art will recognize that the state of the art has progressed to the point where there is little distinction between hardware and software implementations of aspects of apparatuses. Those having ordinary skill in the art will appreciate that there are various vehicles by which processes and/or systems described herein can be effected (for example, hardware, software, and/or firmware), and that the preferred vehicle will vary with the context in which the processes are deployed. For example, if an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

The foregoing detailed description has set forth various embodiments of the devices and/or processes via the use of block diagrams, flowcharts, and examples. Insofar as such block diagrams, flowcharts, and examples contain one or more functions and/or operations, it will be understood by those within the art that each function and/or operation within such block diagrams, flowcharts, or examples can be implemented, individually and/or collectively, by a wide range of hardware, software, firmware, or any combination thereof (or means for performing the respective function and/or operation). However, those skilled in the art will recognize that the embodiments disclosed herein, in whole or in part, can be equivalently implemented in standard Integrated Circuits, via Application Specific Integrated Circuits (ASICs), as firmware, or as virtually any combination thereof, and that designing the circuitry and/or writing the code for the software or firmware would be well within the skill of one of ordinary skill in the art in light of this disclosure.

The exemplary embodiments described above may be combined in a variety of ways with each other. Furthermore, the dimensions, shapes, sizes, and number of the various pieces illustrated in the figures may be adjusted from that shown.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic. As used in this disclosure, “in communication” includes the situations where two pieces abut each other, are connected to each other, engage each other, and integrally formed together as one piece.

Although the present invention has been described in terms of particular embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

We claim:

1. A method for determining the time until a food reaches a desired temperature, said method comprising:
receiving the desired temperature,
determining a current temperature for the food,
measuring a change in temperature as a function of time,
using the temperature change to predict the time until the food reaches the desired temperature, and
starting a timer from the predicted time.

2. The method according to claim 1, further comprising notifying a user when the timer reaches zero.

3. The method according to claim 1, further comprising when the current temperature of the food reaches the desired temperature, notifying the user.

4. The method according to claim 1, wherein repeating the determining, measuring, and using steps at predetermined intervals.

5. The method according to claim 1, wherein repeating the determining, measuring, and using steps continually until the desired temperature is reached.

6. The method according to claim 1, wherein repeating the determining, measuring, and using steps at predetermined intervals until the timer reaches a threshold time remaining.

7. The method according to any one of claims 1, wherein the measuring step uses the formula
t_{R}=(T_{D}−T₂)/(T₂−T₁)*(t₂−t₁)
t_{R} equals the cooking time to reach the desired temperature, T_{D} represents the desired temperature, T₁ equals the temperature at the start of the sampling, T₂ equals the temperature at the end of the sampling with t₁, and t₂ equaling the respective times of the temperature sampling.

8. The method according to any one of claims 1, further comprising displaying the timer on a display.

9. A method for determining the time until a food reaches a desired temperature, said method comprising:
receiving the desired temperature,
determining a current temperature for the food,
measuring a change in temperature as a function of time,
using the temperature change to predict the time until the food reaches the desired temperature,
starting a timer from the predicted time, and
repeating the determining, measuring, and using steps at least one time.

10. The method according to claim 9, wherein the temperature change for the first measurement occurs from about 90 degrees to 100 degrees.

11. The method according to claim 9, wherein repeating the determining, measuring, and using steps after a predetermined amount of temperature change.

12. The method according to claim 9, further comprising notifying the user when the current temperature of the food reaches the desired temperature.

13. The method according to claim 9, wherein repeating the determining, measuring, and using steps at predetermined intervals.

14. The method according to claim 9, wherein repeating the determining, measuring, and using steps continually until the desired temperature is reached.

15. The method according to any one of claims 9, wherein the measuring step uses the formula
t_{R}=(T_{D}−T₂)/(T₂−T₁)*(t₂−t₁)
t_{R} equals the cooking time to reach the desired temperature, T_{D} represents the desired temperature, T₁ equals the temperature at the start of the sampling, T₂ equals the temperature at the end of the sampling with t₁, and t₂ equaling the respective times of the temperature sampling.

16. The method according to claim 1, further comprising notifying a user when the timer reaches zero.

17. A system comprising:
a probe having a temperature sensing component;
a display;
a thermometer circuitry in communication with said temperature sensing component and coupled to said display, said thermometer circuitry having
means for receiving a desired temperature,
means for calculating a current temperature,
means for determining a time remaining to cook based upon a temperature rate change, and
means for controlling said display to show time remaining to cook; and
an interface coupled to said thermometer circuitry.

18. The system according to claim 15, wherein said display includes a desired temperature area, a current temperature area, and a timer.

19. The system according to claim 15, wherein said interface includes a toggle between Celsius and Fahrenheit, a power switch, and a toggle between hours:minutes and minutes:seconds.

20. The system according to claim 15, wherein said interface includes a toggle between Celsius and Fahrenheit, a power switch, a toggle between hours:minutes and minutes:seconds, and a toggle between a timer and time remaining to cook.
